(19) United States
(12) Patent Application Publication
(10) Pub. No.: US 2008/0221437 A1
(43) Pub. Date: Sep. 11, 2008

(54) STEERABLE SNARE FOR USE IN THE COLON AND METHOD FOR THE SAME

(76) Inventors: Mark A. AGRO, Mendon, MA (US); Michael S. H. Chu, Brookline, MA (US); Robert F. Rioux, Ashland, MA (US); Ashley Seehusen, Newton, MA (US); Vincent A. Turturro, Bolton, MA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 12/043,669

(22) Filed: Mar. 6, 2008

Related U.S. Application Data
(60) Provisional application No. 60/893,917, filed on Mar. 9, 2007.

(51) Int. Cl. A61B 5/05; A61B 17/24

(52) U.S. Cl. 600/424; 606/113

(57) ABSTRACT

An apparatus comprises a snare and an actuator. The snare is formed of a material having an opacity complementary to a predetermined imaging system that is positionable external to a patient. The actuator is configured to steer the snare in response to an image of at least a portion of a colon of the patient. The image is generated by the predetermined imaging system.

### CROSS-REFERENCE TO RELATED APPLICATIONS

This application claims priority to U.S. Provisional Application Ser. No. 60/893,917, entitled “Steerable Snare for Use in the Colon and Method for the Same,” filed Mar. 9, 2007; which is incorporated herein by reference in its entirety.

### BACKGROUND

The invention relates generally to medical devices for use in conjunction with a virtual colonoscopy procedure, and more particularly, to methods for thermally treating undesirable growths.

Colorectal cancer is one of the leading causes of deaths from malignancy in the United States, with only lung cancer causing more deaths annually. Colon cancer can be prevented because it usually begins as a benign polyp that grows slowly for several years before becoming cancerous. If polyps are detected and removed, the risk of developing colon cancer is significantly reduced.

Unfortunately, widespread colorectal screening and preventive efforts are hampered by several practical impediments, including limited resources, methodologic inadequacies, and poor patient acceptance leading to poor compliance. Moreover, some tests, such as the fecal occult blood test (FOBT) fail to detect the majority of cancers and pre-cancerous polyps. Additionally, since a sigmoidoscopy only examines a portion of the colon, it also misses many polyps that occur in the remainder of the colon. The accuracy of other tests, such as the barium enema, vary and are not always reliable.

A technique for detecting colorectal cancer using helical computed tomography (CT) to create computer simulated intraluminal flights through the colon was proposed as a novel approach for detecting colorectal neoplasms by Vining D J, Shifrin R Y, Grishaw E K, Liu K, Gelfand D W, Virtual colonoscopy (Abst), Radiology Scientific Prgm 1994; 193(P):446. This technique was first described by Vining et al. in an earlier abstract by Vining D J, Gelfand D W, Noninvasive colonoscopy using helical CT scanning, 3D reconstruction, and virtual reality (Abst), SGR Scientific Program, 1994. This technique, referred to as “virtual colonoscopy”, requires a cleansed colon insufflated with air, a helical CT scan of approximately 30 seconds, and specialized three-dimensional (3D) imaging software to extract and display the mucosal surface. The resulting endoluminal images generated by the CT scan are displayed to a medical practitioner for diagnostic purposes.

There have been several advances in virtual colonoscopy that have improved the imaging techniques, making it a more viable and effective screening option. One advantage of using a virtual colonoscopy as a screening process is the elimination of the invasiveness of a traditional colonoscopy. Traditional colonoscopies are preformed using a colonoscope that has a relatively large diameter (i.e., sufficient to form a seal with the anus) that includes, among other instruments, a scope, multiple lumens for introducing gas and/or liquid, and a working channel for introducing a snare or similar device into the colon. With such a device, there is a risk of straightening and/or perforating the colon because of its relative inflexibility and size.

Another advantage of the virtual colonoscopy procedure is the elimination of the preparation process associated with a traditional colonoscopy. The typical preparation process involves the use of strong laxatives to purge any fecal waste from the colon. Such a process is extremely uncomfortable and is often cited as one of the least desirable parts of the whole procedure. Complete purging is not necessary with the virtual colonoscopy procedure. Rather, a fecal contrasting agent is used to facilitate digital subtraction of any residual feces from the virtual image.

During the procedure, the patient lies on the CT scan area. A thin tube (approximately the diameter of a rectal thermometer) is placed in the rectum, through which gas is introduced into the colon. The gas is necessary to distend the bowel allowing any polyps to stand out from the normal surface. The patient holds their breath while the machine sweeps over the abdomen. The procedure is repeated with the patient lying on their stomach. The whole procedure takes approximately ten minutes.

In addition to CT scan imaging modalities, magnetic resonance imaging (MRI) can also be used to perform the virtual colonoscopy. When using MRI, only certain MRI-compatible tools can be utilized (i.e., tools with only slight ferromagnetic properties).

Even though the virtual colonoscopy is largely non-invasive as a screening process, a need still exists for non-invasive and minimally-invasive devices and methods for treating the colon (e.g., removing polyps) in the event the virtual colonoscopy identifies a problem area within the colon that merits further evaluation or treatment.

For example, during conventional colonoscopies, polyps are removed using a wire-loop snare or similar device that slices the polyp from the wall of the colon. Such a technique is not effective for broad-base polyps or multiple polyps concentrated in a small area due to the excessive bleeding that could result as well as the increased risk of perforation.

What is needed is a minimally-invasive method of removing polyps in the colon without the use of cutting tools such as polyp snares.

### SUMMARY OF THE INVENTION

In one embodiment, an apparatus comprises a snare and an actuator. The snare is formed of a material having an opacity complementary to a predetermined imaging system that is positionable external to a patient. The actuator is configured to steer the snare in response to an image of at least a portion of a colon of the patient. The image is generated by the predetermined imaging system.

In another embodiment, an image of at least a portion of a colon of a patient is generated with an imaging system entirely external to the patient. A surgical instrument is disposed proximate to a polyp in the colon of the patient. The surgical instrument has a snare. The snare is steered to a location proximate the polyp based on the image of the colon.

### BRIEF DESCRIPTION OF THE DRAWINGS

FIG. 1 depicts a block diagram of a polyp removal system, according to an embodiment of the invention.

FIG. 2 depicts a side view of a surgical device, according to an embodiment of the invention.

FIG. 3 depicts a side view of an example of an RF snare that is a bipolar device, according to an embodiment of the invention.

FIG. 4 depicts a perspective view of a cross-sectional cut-away of the surgical device shown in FIG. 2 and taken along the line 4-4 in FIG. 2.

FIG. 5 depicts an example of an application of the polyp removal system while disposed within a patient's colon.

FIG. 6 depicts a system block diagram of a polyp removal system, according to another embodiment of the invention.

### DETAILED DESCRIPTION

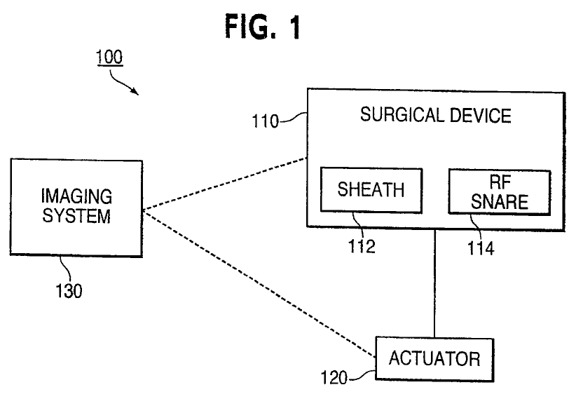

FIG. 1 depicts a block diagram of a polyp removal system, according to an embodiment of the invention. As shown in FIG. 1, the polyp removal system 100 includes a surgical device 110, an actuator 120 and an imaging system 130. The surgical device includes a snare 114.

The snare 114 can be made of a material having an opacity complimentary to imaging system 130. For example, in an embodiment where imaging system 130 is a fluoroscopic imaging system, snare 114 can be made of a material that is opaque to fluoroscopic imaging and electrically conductive such as a metal, a filled plastic or a combination of metal and filled plastic. For example, a suitable metal can be stainless steel or a metal filled with nitinol such as that described in U.S. Pat. No. 6,290,721, which is incorporated herein by reference in its entirety. For another example, a suitable filled plastic can be a plastic rendered opaque with bismuth trioxide, gold powder, platnum powder, bismuth oxychloride, iridium powder, tungsten, silver powder and/or barium sulfate. In another embodiment where imaging system 130 is a magnetic resonance imaging (MRI) system, snare 114 can be made of a material that is opaque to magnetic resonance such as plastic with slight ferromagnetic properties to avoid movement and/or image distortion from an MRI.

Although the snare 114 is described and shown herein as a close loop, it should be understood that other similar devices are possible including, for example, biopsy forceps, biopsy needles, resecting devices, resecting wires, morcellators, etc.

In yet another embodiment, imaging system 130 can include a virtual computer tomography (CT) system in combination with a fluoroscopic imaging system or a MRI system; in such an embodiment, snare 114 can be made of a material as described above. The physician can use virtual CT system to approximate an initial placement of the snare 114, and then the fluoroscopic imaging system or MRI imaging system can be used to confirm and/or adjust the proper location of the RF snare 113. In this embodiment, the location and orientation of snare 114 can be coordinated with the images produced with the virtual CT system of imaging system 130. Thus, the images displayed by the imaging system 130 can be updated as the physician changes the position of the snare 114. In this manner, the use of to a non-virtual imaging system is minimized, thereby reducing the time of the procedure.

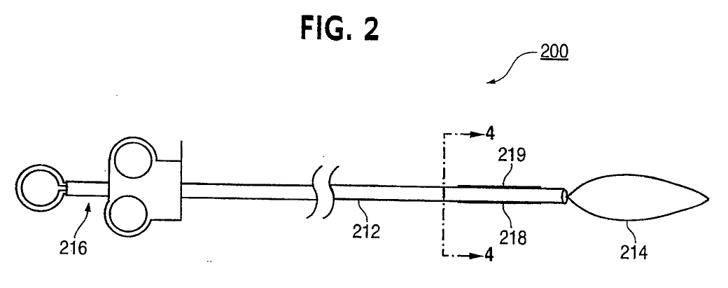

FIG. 2 depicts a side view of a surgical device, according to an embodiment of the invention. Surgical device 210 includes sheath 212, RF snare 214, actuator 216 and wires 218 and 219. Actuator 216 is coupled to RF snare 214 and wires 218 and 219. Wires 218 and 219 are also coupled to a distal end portion of sheath 212. RF snare 214 is coupled to an electrical source (not shown), which provides an electrical current to RF snare 214.

Surgical device 210, for example, can be used directly without being combined with an endoscope. In other words, surgical device 210 is made of appropriate materials that allow the distal end portion to be disposed within a patient and moved to an appropriate position, for example, within the patient's colon. For example, the sheath 212 can be made from a material such as plastic or rubber that is sufficient flexible so that the surgical device 210 can be inserted into the colon of the patient without straightening the colon. In addition, the sheath 212 can have an outer diameter, for example, of 3 mm or less. The construction and size of such a surgical device 210 allows for less patient discomfort than would be the case with a larger endoscope.

In another embodiment, a low-profile guide device (not shown) can be used in conjunction with the surgical device 210. In such an embodiment, the a low-profile guide sheath or guidewire can be delivered to the treatment site prior to delivery of surgical device 210. Once delivered to the treatment site, the low-profile guide device can be used to lead the surgical device 210 to the treatment site.

Surgical device 210 can be configured such that the distal end portion of sheath 212 can be steered and snare 214 can be rotated to a desired position relative to the colon wall. For example, a physician can steer the distal end portion of surgical device 210 in one direction or the opposite direction via wires 218 and 219, and can rotate snare 214 via actuator 216. The distal end portion of the sheath 212 can be steered, for example, in a lateral or side-to-side direction with respect to the length of the of the surgical device 210. Through this combination of steering and rotating, the snare 214 can be positioned about a polyp within the colon. The following references provide examples of a rotatable snare and each reference is incorporated herein by reference: U.S. Pat. Nos. 6,162,209; 6,235,026; 6,409,727; 6,517,539; 6,554,942; 6,602,262; 6,911,032; and U.S. Patent Application Publication Nos. 2003/0105488, 2004/0181243, 2004/0199052, 2005/0113845, 2005/0119527, 2005/0124912, and 2005/0131279.

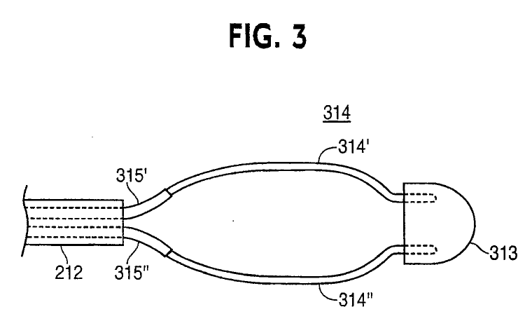

The RF snare can be, for example, a bipolar device. FIG. 3 depicts a side view of an example of an RF snare that is a bipolar device, according to an embodiment of the invention. As shown in FIG. 3, the RF snare 314 includes a first RF snare portion 314′ and a second RF snare portion 314″, which is electrically isolated from the first RF snare portion 314′ by insulation cap 313. More specifically, first RF snare portion 314′ and second RF snare portion 314″ are disposed within insulation sleeves 315′ and 315″, respectively. Insulation sleeves 315′ and 315″ electrically insulate first RF snare portion 314′ and second RF snare portion 314″ from sheath 212. In an alternative embodiment, the sheath can have, for example, two electrically isolated lumens, one for the first RF snare portion and one for the second RF snare portion; in such an alternative embodiment, the sleeves are not necessary because the sheath provides the electrical isolation.

Distal ends of first RF snare portion 314′ and second RF snare portion 314″ are disposed within an insulation cap 313. Insulation cap 313 electrically insulates first RF snare portion 314′ from second RF snare portion 314″. Thus, when current is applied to the RF snare 314, an RF field is produced between the exposed portions of first RF snare portion 314′ and second RF snare portion 314″. This RF field can ablate tissue disposed between first RF snare portion 314′ and second RF snare portion 314″ as described below. In an alternative embodiment, the RF snare can be a monopolar device configured for use with a grounding pad (not shown) on the exterior of a patient. In yet another embodiment, the snare is not energized.

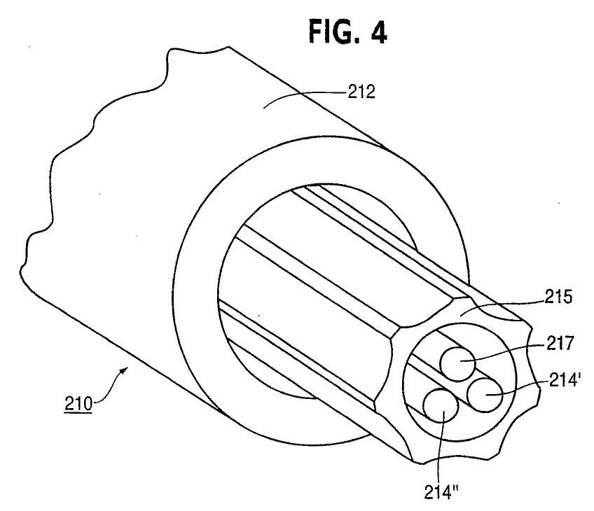

FIG. 4 depicts a perspective view of a cross-sectional cut-away of the surgical device shown in FIG. 2 and taken along the line 4-4 in FIG. 2. The portion of surgical device 210 shown in FIG. 4 includes sheath 212, an inner catheter 215, RF snare portions 214′ and 214″, and shaft 217. The distal end portion of shaft 217 is operationally coupled to actuator 216 (shown in FIG. 2). The proximate end portion of shaft 217 is fixedly coupled to the distal end portions of RF snare portions 214′ and 214″. The proximate end portion of shaft 217 and the distal end portions of RF snare portions 214′ and 214″ are fixedly coupled to inner catheter 215. Inner catheter 215 is rotatably disposed within sheath 212.

The proximate end portion of shaft 217 can be fixedly coupled, for example, to the distal end portions of RF snare portions 214′ and 214″ via a bearing (not shown) within inner catheter 215. Alternatively, the proximate end portion of shaft 217 can fixedly coupled, for example, to inner catheter 215 by a first bearing (not shown) within inner catheter 215, and the distal end portions of RF snare portions 214′ and 214″ can be fixedly coupled, for example, to inner catheter 215 by a second bearing (not shown) within inner catheter 215.

As the actuator 216 is actuated with a rotational motion, this rotational motion is translated to shaft 217 and, consequently, to inner catheter 215 and RF snare portions 214′ and 214″. In general, this embodiment allows the snare 212 to rotate via actuator 216 without, for example, an undesired “whipping effect” where the snare 212 rapidly rotates with little or no control of the position of the snare 212. Because the medical device 210 may be disposed within a patient along a tortuous and lengthy path, it is desirable that rotation of the snare 214 via the actuator 216 is effective and controlled. The inner catheter 215 contacts and rotates against the inner surface of sheath 212 while maintaining the relative position of shaft 217 and RF snare portions 214′ and 214″ within inner catheter 215. U.S. Pat. Nos. 6,840,900 and 6,454,702 disclose examples of an inner catheter similar to the inner catheter 215, and each is incorporated herein by reference.

The embodiment shown in FIG. 4 is one of many possible embodiments. For example, the shape of the inner catheter can be a shape other than the star-like shape of inner catheter 215 shown in FIG. 4. Alternatively, the inner catheter arrangement can be embodied by any type of swivel structure such as a ball-and-socket arrangement. Various possible alternative embodiments are disclosed in U.S. Patent Application Publication 2005/0113845, entitled “Self-Orienting Polypectomy Snare Device,” the disclosure of which is incorporated herein by reference. Other alternative embodiments are also disclosed in U.S. Pat. No. 6,602,262, entitled “Medical Device Having Linear to Rotation Control,” the disclosure of which is incorporated herein by reference.

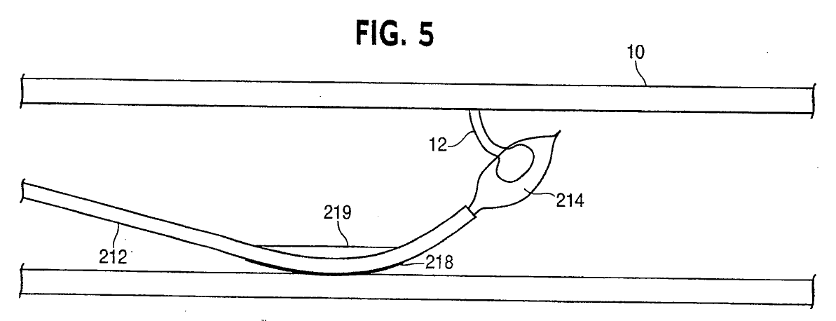

FIG. 5 depicts an example of an application of the polyp removal system while disposed within a patient's colon. The patient's colon 10 is shown in FIG. 5 in a cut-away view and includes a polyp 12 extending from the wall of the colon 10. The distal end of the surgical device 210 can be disposed within the appropriate location within the colon 10. As discussed above, a physician can steer the distal end portion of surgical device 210 in one direction or the opposite direction via wires 218 and 219. RF snare 214 can be positioned about the stalk of polyp 12 through manipulation of actuator 216. More specifically, once the distal end of surgical device 210 is positioned near the polyp 12, the actuator 216 can be actuated to extend RF snare 214 from the sleeve 212. Once extended from sleeve 212, the RF snare 214 can be positioned about a portion of polyp 12 (e.g., the narrow stalk of polyp 12). Then, the RF snare 214 can be electrically activated while being closed about polyp 12.

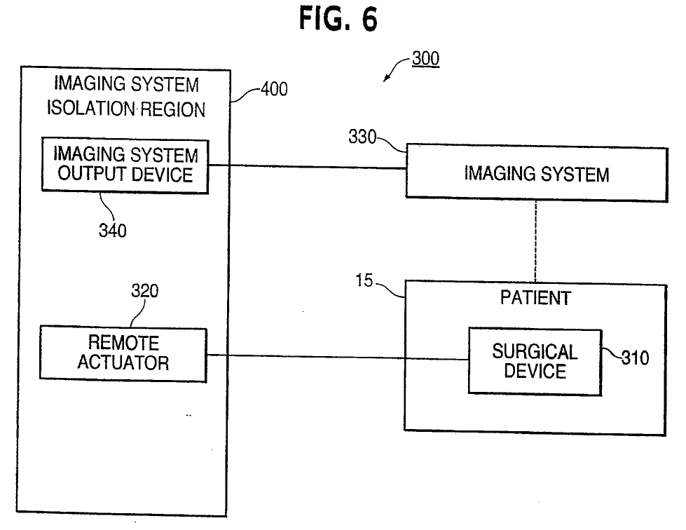

FIG. 6 depicts a system block diagram of a polyp removal system, according to another embodiment of the invention. As shown in FIG. 6, the polyp removal system 300 includes a surgical device 310, a remote actuator 320, an imaging system 330 and an imaging system output device 340. Remote actuator 320 is coupled to and controls surgical device 310. Imaging system output display 340 is a display unit for imaging system 330 such as, for example, a video display. Patient 15 can be positioned within polyp removal system 300 such that surgical device 310 is appropriately disposed within patient 15 for polyp removal and imaging system 330 to obtain images of the patient 15 and surgical device 310.

Imaging system output device 340 and remote actuator 320 are disposed within an imaging system isolation region 400. Imaging system isolation region 400 electromagnetically isolates imaging system output device 340 and remote actuator 320 from imaging system 330. For example, where imaging system 330 is an MRI system, which can interact with any metal device, imaging isolation region 400 isolates imaging system output device 340 and remote actuator 320 from imaging system 330. In such a case, imaging isolation region 400 can be, for example, an isolation chamber or room that prevents the MRI system of imaging system 330 from imaging people and equipment within imaging isolation region 400.

Remote actuator 320 is remote from surgical device 310 in the sense that actuator 320 is separated from surgical device 310 by imaging system isolation region 400. Remote actuator 320 can be, for example, a robotic system by which a physician can control surgical device 310. Where the surgical device 310 is similar to the surgical device 110 shown in FIGS. 2-4, a physician located within imaging system isolation region 400 can remotely control, for example, the position of the distal end portion of the sheath of surgical device 310, the position and orientation of the RF snare of surgical device 310 and the electrical power provided to the RF snare.

### Conclusion

While various embodiments of the invention have been described above, it should be understood that they have been presented by way of example only, and not limitation. While various embodiments have been particularly shown and described, it will be understood that various changes in form and details may be made. For example, although a particular type of actuator is shown in FIG. 2, other types of actuators also possible. Such alternative actuators include, for example, other types of manual devices controlled by a medical practitioner and non-manual devices such as machine-controlled actuators. Such a machine-controlled actuator can be operated in conjunction with the output of the imaging system.

Any of the above-discussed surgical devices can further include a visualization system internal to the surgical device. In one embodiment, for example, the surgical device can include an internal optical fiber that is part of an optical system for visualization. In such an embodiment, the external imaging system can target tissue and the optical fiber within the surgical device can provide higher resolution information about the tissue site to minimize treating or affecting healthy tissue during the procedure.

In other embodiments, the polyp removal system can be used in conjunction with multiple devices under virtual colonoscopy. For example, a resecting device can include a resecting razor and as tapler for closing the resecting site. Other embodiments include but are not limited to needles, biopsy forceps, snares, staplers, fasteners, suturing systems and other diagnostic and therapeutic tools.

What is claimed is:

1. An apparatus, comprising:
a snare being formed of a material having an opacity complementary to a predetermined imaging system that is positionable external to a patient; and
an actuator configured to steer the snare in response to an image of at least a portion of a colon of the patient, the image being generated by the predetermined imaging system.

2. The apparatus of claim 1, further comprising:
a sheath coupled to the snare, the sheath has a diameter no greater than 3 mm.

3. The apparatus of claim 1, further comprising:
a sheath coupled to the snare, the sheath being made from a material sufficiently flexible to be inserted into the colon of the patient without straightening the colon of the patient.

4. The apparatus of claim 1, wherein:
the actuator is configured to steer the snare to a location about a polyp within the colon of the patient in response to an image of the patient, the polyp and the surgical instrument.

5. The apparatus of claim 1, wherein:
the actuator is a remote actuator disposed within an imaging system isolation region; and
the predetermined imaging system is a real-time imaging system.

6. The apparatus of claim 1, wherein the actuator is a first actuator, the apparatus further comprising:
a sheath coupled to the snare; and
a second actuator configured to rotate the snare relative to the sheath, the second actuator being different from the first actuator.

7. The apparatus of claim 1, further comprising:
a sheath coupled to the snare, a distal end portion of the sheath being coupled to the actuator, the actuator configured to steer the snare by moving the distal end portion of the sheath.

8. An apparatus, comprising:
a surgical instrument having a snare and an actuator configured to steer the snare; and
a predetermined imaging system that is positionable external to a patient, the imaging system configured to externally produce an image of at least a portion of a colon of a patent.

9. The apparatus of claim 8, further comprising:
a sheath coupled to the snare, the sheath of the surgical instrument has a diameter no greater than 3 mm.

10. The apparatus of claim 8, further comprising:
a sheath coupled to the snare, the sheath of the surgical instrument being made from a material sufficiently flexible to be inserted into the colon of the patient without straightening the colon of the patient.

11. The apparatus of claim 8, wherein:
the predetermined imaging system is coupled to the actuator of the surgical instrument, the actuator configured to steer the snare in response to the image of the colon and the surgical instrument.

12. The apparatus of claim 8, wherein:
the predetermined imaging system configured to externally produce the image of the colon without the surgical instrument being disposed within the patient, the image being a virtual colonoscopy image.

13. The apparatus of claim 8, wherein:
the actuator is disposed within an imaging system isolation region; and
the imaging system is a real-time imaging system.

14. The apparatus of claim 8, wherein the actuator is a first actuator, the apparatus further comprising:
a sheath coupled to the snare; and
a second actuator configured to rotate the snare relative to the sheath, the second actuator being different from the first actuator.

15. The apparatus of claim 8, further comprising:
a sheath coupled to the snare, a distal end portion of the sheath being coupled to the actuator, the actuator configured to steer the snare by moving the distal end portion of the sheath.

16. A method comprising:
generating an image of at least a portion of a colon of a patient with an imaging system entirely external to the patient;
disposing a surgical instrument proximate to a polyp in the colon of the patient, the surgical instrument having a snare; and
steering the snare to a location proximate the polyp based on the image of the colon.

17. The method of claim 16, further comprising:
generating an image of at least a portion of the colon, the polyp and the snare with the imaging system; and
steering the snare to a location about the polyp based on the image of the portion of the colon, the polyp and the snare.

18. The method of claim 16, the imaging system being a first imaging system, the method further comprising:
generating an image of at least a portion of the colon, the polyp and the snare with a second imaging system entirely external to the patient, the second imaging system being different from the first imaging system; and
steering the snare to a location about the polyp based on the image of the portion of the colon, the polyp and the snare.

19. The method of claim 16, wherein:
the surgical instrument has a diameter no greater than 3 mm.

20. The method of claim 16, wherein:
the surgical instrument being made from a material sufficiently flexible such that the disposing the surgical instrument is performed without straightening the colon of the patient.

21. The method of claim 16, wherein:
the generating the image being performed without the surgical instrument being disposed within the patient, the image being a virtual colonoscopy image.

22. The method of claim 16, wherein:
the disposing the surgical instrument being controlled by a remote actuator disposed within an imaging system isolation region; and
the generating the image being performed in substantially a real time.
